SEQUENCE LISTING (4 sequences)

SEQ ID NO: 1 - DNA, 22 bp, Homo sapiens
  hsa-miR-615-5p
ggggguccccggugcucggauc

SEQ ID NO: 2 - DNA, 24 bp, Homo sapiens
  hsa-miR-125a-5p
ucccugagacccuuuaaccuguga

SEQ ID NO: 3 - DNA, 22 bp, Homo sapiens
  hsa-let-7a-5p
ugagguaguagguuguauaguu

SEQ ID NO: 4 - DNA, 22 bp, Homo sapiens
  hsa-let-7b-5p
ugagguaguagguugugugguu

(19) United States
(12) Patent Application Publication
(10) Pub. No.: US 2019/0024183 A1
(43) Pub. Date: Jan. 24, 2019

(54) DIFFERENTIAL DIAGNOSIS IN GLIOBLASTOMA MULTIFORME

(71) Applicant: Helmholtz Zentrum München - Deutsches Forschungszentrum für Gesundheit und Umwelt (GmbH), Neuherberg (DE)

(72) Inventors: Claus Belka, München (DE); Karim-Maximilian Niyazi, München (DE); Kristian Unger, München (DE); Horst Zitzelsberger, Oberschleißheim (DE); Adriana Pitea, München (DE); Michel Mittelbronn, Freiburg (DE)

(21) Appl. No.: 16/063,175

(22) PCT Filed: Dec. 22, 2016
(86) PCT No.: PCT/EP2016/082411

§ 371 Date: Jun. 15, 2018

(30) Foreign Application Priority Data
Dec. 23, 2015 (EP) 15202328.9

(51) Int. Cl. C12Q 1/6886

(52) U.S. Cl. CPC C12Q 1/6886; C12Q 1/6806

(57) ABSTRACT

The present invention relates to a kit (a) comprising or (b) consisting of means for detecting and/or quantifying one, two, three or all four of the following miRNAs: hsa-miR-615-5p; hsa-miR-125a-5p; hsa-let-7a-5p; and hsa-let-7b-5p.

The present invention relates to a kit (a) comprising or (b) consisting of means for detecting and/or quantifying one, two, three or all four of the following miRNAs: hsa-miR-615-5p; hsa-miR-125a-5p; hsa-let-7a-5p; and hsa-let-7b-5p.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Malignant gliomas account for approximately 70% of primary brain tumors diagnosed in adults. Median age at diagnosis is 64 years with men being more frequently affected than women (Fisher et al., Neurol Clin. 2007; 25:867-90, vii.). Amongst all gliomas, glioblastoma multiforme (GBM) is the most common and aggressive form (Louis et al., Acta Neuropathol. 2007; 114:97-109). State-of-the-art treatment of GBM comprises surgical resection and adjuvant radiochemotherapy followed by maintenance chemotherapy. Implementation of temozolomide (TMZ) into the radiochemotherapeutic regime improved 2-year survival rates of patients with newly diagnosed malignant glioma (mainly GBM) from 11% to 27%, 3-year survival rates from 4% to 16%, and 5-year survival rates from 2% to 10% (Stupp et al., Lancet Oncol. 2009; 10:459-66; Stupp et al., N Engl J Med. 2005; 352:987-96). Recent prospective phase III trials (AVAglio & RTOG) evaluated the efficacy of TMZ-based radiochemotherapy in combination with the anti-angiogenic agent bevacizumab as first-line therapy. Unfortunately, these trials failed to show improvements in overall survival, but significant and marginally significant benefits in progression-free survival were observed (Chinot et al., N Engl J Med. 2014; 370(8):709-722; Gilbert et al., N Engl J Med. 2014; 370:699-708). Integrin targeting by cilengitide also failed to provide an additional benefit over classical TMZ-based radiochemotherapy according to the EORTC/NCIC26981/22981-NCIC CE3 trial (Stupp et al., Lancet Oncol. 2014; 15:1100-8). Thus, the results of molecularly targeted treatment approaches so far confirm TMZ-based radiochemotherapy as standard treatment for GBM.

The advent of high precision treatment planning algorithms, image-guided dose application, and novel irradiation qualities, as well as the introduction of TMZ have provided clear therapeutic improvements for primary and recurrent GBM (Niyazi et al., Radiat Oncol. 2014; 9:299; Niyazi et al., Radiat Oncol. 2013; 8:287; Niyazi et al., Radiat Oncol. 2011; 6:177). However, prognosis of most GBM patients still remains dismal with a high rate of local recurrence, emphasizing the clear need for further optimization (Niyazi et al., Radiat Oncol. 2014; 9:299; Weber et al., Radiother Oncol. 2009; 93:586-92). At present, several strategies are being followed in this regard: Firstly, more elaborate imaging techniques as well as improved image-guidance during radiotherapy are being tested (Cyran et al., Radiat Oncol. 2014; 9:3; Gilbert et al. RTOG 0625: A phase II study of bevacizumab with irinotecan in recurrent glioblastoma (GBM). 2009; Niyazi et al., Radiother Oncol. 2011; 98:1-14; Stall et al., Radiat Oncol. 2010; 5:5.). Secondly, various molecularly designed substances are undergoing pre-clinical and clinical testing for their therapeutic efficacy in combination with radio(chemo)therapy (Beal et al., Radiat Oncol. 2011; 6:2; Flieger et al., J Neurooncol. 2014; 117:337-45; Niyazi et al., Int J Radiat Oncol Biol Phys. 2010; Stupp et al., J Clin Oncol. 2010; 28:2712-8; Welsh et al., Radiat Oncol. 2009; 4:69.). These targeted treatment approaches require molecular stratification of patients in order to identify the subgroups that can benefit most from a given strategy. Classical radiochemotherapy also displays wide inter-individual differences in terms of response and survival rates (Frenel et al., Bulletin Du Cancer. 2009; 96:357-367). Accordingly, numerous efforts are undertaken in order to characterize the molecular mechanisms orchestrating therapy sensitivity and resistance and to identify prognostic and predictive markers.

So far, only few prognostic factors have been defined for GBM, including age and Eastern Cooperative Oncology Group (ECOG) score. In addition, involvement of the subventricular zone and extent of resection are known to be of negative prognostic values (Adeberg et al., Radiation Oncology (London, England). 2014; 9:95.). More recently, the first molecular markers have been established. In this regard, methylation of the O6-methylguanine DNA-methyltransferase (MGMT) promoter region was recognized to be of positive predictive value for the efficacy of TMZ-based radiochemotherapy, and molecular profiling of long-term survivors disclosed the positive prognostic value of a proneural-like expression pattern linked to mutations in the genes encoding for isocitrate dehydrogenases 1/2 (IDH1/2) (Hegi et al., J Clin Oncol. 2008; 26:4189-99; Kim Hak Jae et al., Radiation oncology (London, England). 2012; 7:39; Reifenberger et al., Int J Cancer. 2014; 135:1822-31).

During the last years, microRNAs (miRNAs) have increasingly received attention. With a high degree of promiscuity miRNAs target and regulate several mRNA species encoding for proteins involved in various signaling pathways (Ha Minju, Kim V. Narry, Nature Reviews Molecular Cell Biology. 2014; 15:509-524). Accumulating evidence indicates that miRNA expression signatures can serve as biomarkers for diagnosis and risk assessment of diverse malignancies, including GBM (Cheng Wen, et al. Oncotarget. 2015.; De Smaele et al., Brain Res. 2010; 1338:100-11; Gao et al., Oncogene. 2014; 0; Jiang et al., American Journal of Pathology. 2010; 177:29-38; Manterola et al., Neuro-Oncology. 2014:not218; Sana et al., Carcinogenesis. 2014; 35:2756-2762; Shou et al., Experimental and Therapeutic Medicine. 2015; 9:167-171; Zhang et al., PLoS ONE. 2014; 9:e96908). Given that the available prognostic markers can segregate GBM patients only to a limited extent, additional and better markers and/or signatures are sought for. Moreover, in many instances the prior art studies did not select GBM patients which receive the standard-of-care treatment which limits their usefulness.

The technical problem underlying the present invention can be seen in the provision of alternative or improved methods for stratifying GBM patients. This problem is solved by the subject-matter of the claims.

Accordingly, the present invention, in a first aspect, relates to a kit (a) comprising or (b) consisting of means for detecting and/or quantifying one, two, three or all four of the following miRNAs: hsa-miR-615-5p; hsa-miR-125a-5p; hsa-let-7a-5p; and hsa-let-7b-5p.

The term “microRNA”, abbreviated “miRNA”, has its art-established meaning. It designates small RNAs which are encoded by the genome of an animal or the human genome. MiRNAs do not encode proteins or peptides. The understanding of their function is still emerging. Generally speaking, they frequently play a role in gene regulation including gene silencing. The size of miRNAs is between a few dozen and a few hundred nucleotides. In terms of structure, miRNAs in many instances are hairpin molecules, i.e. they consist of a single strand which falls back upon itself, thereby giving rise to a stem and a loop, wherein within the stem there may be bulges and/or non-paired nucleotides. MiRNAs are typically the result of the processing of pre-miRNAs which are larger in size and more complex in structure (several loops and several stems). A recent review of the field of miRNAs is Minju & Narry, loc. cit.

The four miRNAs, namely hsa-miR-615-5p; hsa-miR-125a-5p; hsa-let-7a-5p; and hsa-let-7b-5p, are the mature forms of the corresponding precursors. The nomenclature given conforms with that published by the mirBase consortium (Griffiths-Jones S, Grocock R J, van Dongen S, Bateman A, Enright A J. Nucl. Acids Res. 2006 34:D140-D144). In more detail, “hsa” refers to the species (i.e. homo sapiens) of origin of the sequence, the capital letter “R” indicates the mature form of the respective miRNAs, and -5p and -3p, respectively, defines from which part of the precursor stem-loop the miRNA is derived from.

The sequences of the four miRNAs are set forth by SEQ ID NOs: 1 to 4.

As will be apparent from the enclosed examples, the present inventors surprisingly found that a very small set of miRNAs, preferably four miRNAs, may be used to stratify GBM patients in a highly reliable manner. While already each single miRNA is able to statistically significantly predict overall survival of GBM patients, preference is given to the set of the four miRNAs as defined above.

While it is preferred that the kit in accordance with the first aspect contains means for detecting and/or quantifying all four of the miRNAs in accordance with the present invention, the invention also extends to combinations of less than four miRNAs.

In that sense, a particularly preferred combination of three miRNAs is that of hsa-let-7b-5p, hsa-miR-615-5p and hsa-let-7a-5p. Accordingly, a kit in accordance with the first aspect may comprise or consist of means for detecting and/or quantifying these three miRNAs.

A preferred combination of two miRNAs is that of hsa-let-7b-5p and hsa-let-7a-5p. Accordingly, another implementation of the first aspect of the present invention is a kit comprising or consisting of means for detecting and/or quantifying these two miRNAs.

In a preferred embodiment of the kit comprising said means, said kit comprises no means for detecting and/or quantifying miRNAs or RNAs other than one, two, three or all four of hsa-miR-615-5p; hsa-miR-125a-5p; hsa-let-7a-5p; and hsa-let-7b-5p, wherein said kit optionally comprises means for quantifying one or more standard RNAs.

This preferred embodiment clarifies that the open language (“comprising”) allows for the presence of further subject-matter such as vessels, carriers, or manuals, but does not extend to the provision of entire arrays which are designed for assessing expression levels of more than four such as hundreds of miRNAs. In fact, to the extent the kit allows for the presence of means for detecting and/or quantifying RNAs other than the four miRNAs according to the invention, this is confined to means for quantifying one or more standard RNAs. That standard RNAs in turn serve as an internal standard which aids in quantifying the expression levels of the four miRNAs according to the invention. RNAs which qualify as internal standards are generally those which are not regulated differentially. While preference is given to those standard RNAs which are not miRNAs, it is not totally excluded to use also one or more miRNAs as standards, especially to the extent they are not differentially regulated.

Since miRNA regulation is highly dynamic, in general they do not qualify as standards to be used in miRNA expression normalisation. For normalization preferably constitutively expressed small non-noncoding RNAs such as RNU6, SNORD61, SNORD65 and/or SNORD95 are used. These small RNAs are larger in size (45-200 nt) compared to miRNAs (usual range: 21-23 nt) and have been shown to exhibit constant expression. Particularly preferred is the use of SNORD61 for normalization.

Normalization is generally effected by dividing the expression data of the miRNA to be normalized by the expression data of a constitutively expressed RNA.

The kit of the invention may comprise a manual which manual comprises instructions for performing methods of the invention disclosed further below.

Preferred means for detecting and/or quantifying are disclosed further below and include probes and primers specific for the miRNAs of the invention.

In a second aspect, the present invention provides a method of stratifying glioblastoma multiforme (GBM), said method comprising or consisting of determining for a GBM patient one, two, three or all four of the expression levels of miRNAs hsa-miR-615-5p, hsa-miR-125a-5p, hsa-let-7a-5p and hsa-let-7b-5p, wherein (a) the more the expression level of hsa-miR-615-5p is increased and/or the more the expression levels of hsa-miR-125a-5p, hsa-let-7a-5p and hsa-let-7b-5p are decreased, the worse is the prognosis of said patient; (b) determining whether an expression level is increased or decreased is by comparison with a reference state, said reference state being the average expression levels in GBM of hsa-miR-615-5p, hsa-miR-125a-5p, hsa-let-7a-5p and hsa-let-7b-5p, respectively; and (c) diagnostic methods practised on the human or animal body as well as methods for treatment of the human or animal body by surgery are excluded.

The term “stratifying” has its art-established meaning, especially in the sense as it is used in the field of diagnostics and pharmacogenomics. Accordingly, “stratifying GBM” relates to a differential diagnosis of GBM which permits to identify two or more groups or a continuum with regard to a given parameter within GBM patients. From a pharmacogenomics perspective, said stratifying serves to identify subgroups within all GBM patients which subgroups respond favourably to treatment. As will become apparent below, in the present case the diagnostic and the pharmacogenomic aspect of stratifying GBM generally coincide.

Stratifying allows the assignment of GBM patients to two or more groups or ordering them with regard to a given parameter. I.e., stratifying does not necessarily imply the binning of patients. Instead, a given property of patients may scale with the deviation of the expression levels of the miRNAs from the reference state, i.e. the larger the deviation from the reference state, the larger will be the expected value for the given parameter. In accordance with the second aspect of the invention, said parameter is the prognosis for a given GBM patient. The more the expression levels are increased and decreased in accordance with item (a) of the second aspect, the worse is the prognosis of the patient.

The term “prognosis” has its art-established meaning in the field of medicine. In particular, it designates the probable outcome of a patient with regard to measures such as recurrence, overall survival, cancer specific survival and others. In the context of glioblastoma multiforme usually the outcome of interest is overall survival. The median overall survival of glioblastoma multiforme patients is about 14 months, however, overall survival varies very much on the individual level. “Worse prognosis” in accordance with the present invention accordingly defines preferably an expected survival time of the GBM patient under consideration of less than about 14 months. “Average expression levels in GBM” as recited in item (b) of the second aspect accordingly are expression levels which are indicative of an expected survival time of about 14 months. Patients are treated according to standard of care (SOC; see Stupp et al., loc. cit.)

As noted above, the reference state is defined in terms of the average expression levels in glioblastoma multiforme of the four miRNAs of the invention. Ideally, said average expression levels are determined using as many GBM patients as possible. This would be in favor of unbiased sampling. In practical terms, said average expression levels may also be determined for smaller cohorts of GBM patients. For example, if the method of the invention is to be practised at a given site, such as a hospital, said reference state may be defined in terms of the average expression levels for all GBM patients which are treated in said hospital and gave consent to determining the expression levels of the four miRNAs according to the invention.

The following correlation between diagnostic and pharmacogenomic stratification applies. The worse the prognosis of a patient, the less likely it is that any of the above described therapies of GBM has a beneficial effect. If no beneficial effects are to be expected, and giving due consideration to side effects of the given treatment, it may be adequate to dispense with certain forms of treatment altogether. Such a decision, however, can only be made if simple reliable and rapid means of determining the prognosis for a given patient are available. Such means are provided by the present invention.

Preferred smaller sets of miRNAs, in particular sets of two and three miRNAs, respectively, are those which are disclosed in relation to the first aspect herein above.

The determination of expression levels can be done by any suitable method. Such methods include, but are not confined to quantitative PCR for which the primers as disclosed below may be employed.

Alternatively, expression profiling can be done using miRNA arrays. miRNA arrays may be manufactured for the purpose of the present invention in a tailored manner in which case they would contain probes for one, two, three or four of the miRNAs of the invention and optionally probes for one or more standards. Standards are preferably non-differentially regulated RNAs as disclosed herein above. Alternatively, commercially available miRNA arrays may be used. CBC of Heidelberg, Germany, is a manufacturer of miRNA arrays. In accordance with the present invention, it is preferred that the expression data obtained with such arrays (which may comprise probe sets for more than four miRNAs are used to the extent they relate to one, two, three or four miRNAs of the present invention and optionally one or more standard RNAs.

The terms “increased” and “decreased”, respectively, preferably relate to statistically significant increased and statistically significant decreased expression levels of the miRNAs.

Related to the second aspect, the present invention also provides a method of stratifying glioblastoma multiforme (GBM), said method comprising or consisting of determining for a GBM patient one, two, three or all four of the expression levels of miRNAs hsa-miR-615-5p, hsa-miR-125a-5p, hsa-let-7a-5p and hsa-let-7b-5p, wherein (a) the more the expression level of hsa-miR-615-5p is increased and/or the more the expression levels of hsa-miR-125a-5p, hsa-let-7a-5p and hsa-let-7b-5p are decreased, the worse is the prognosis of said patient; and (b) determining whether an expression level is increased or decreased is by comparison with a reference state, said reference state being the average expression levels in GBM of hsa-miR-615-5p, hsa-miR-125a-5p, hsa-let-7a-5p and hsa-let-7b-5p, respectively.

In a preferred embodiment of the method according to the second aspect of the present invention, (i) an expression level of hsa-miR-615-5p which is increased and/or expression levels of hsa-miR-125a-5p, hsa-let-7a-5p and hsa-let-7b-5p which are decreased is/are indicative of bad prognosis; and (ii) an expression level of hsa-miR-615-5p which is decreased and/or expression levels of hsa-miR-125a-5p, hsa-let-7a-5p and hsa-let-7b-5p which are increased is/are indicative of good prognosis.

The term “good prognosis” designates a life-expectancy which is above the average life-expectancy of GBM patients. The term “bad prognosis” designates a life-expectancy which is below the average life-expectancy. The average life-expectancy is determined across all GBM patients. As noted above, where possible, as many patients as available are used for determining the average. In a particular embodiment, the average is determined for a random sample of at least 50, at least 100, at least 200, at least 500 or at least 1,000 GBM patients. These patients may be recruited worldwide and/or may be of Caucasian and/or Japanese origin. Such definitions of “average” apply throughout this specification.

The terms “good prognosis” and “lower-risk GBM” are used equivalently herein. Similarly, the terms “bad prognosis” and “higher-risk GBM” are also used equivalently in this specification.

In a further preferred embodiment of the method of the second aspect, said method does not comprise assessing expression levels of miRNAs or RNAs other than one, two, three or all four of hsa-miR-615-5p; hsa-miR-125a-5p; hsa-let-7a-5p; and hsa-let-7b-5p, wherein said method optionally comprises quantifying the expression level(s) of one or more standard RNAs.

The same notion has been expressed in relation to the kit of the first aspect. It is a key feature of the invention that a surprisingly small set of four miRNAs or yet smaller sets as disclosed above provide an excellent predictor of the prognosis of GBM patients. Accordingly, to the extent expression levels of further RNAs than the four specific miRNAs of the invention are to be quantified, this is confined to standard RNAs.

In a third aspect, the present invention provides a method of diagnosing higher-risk GBM or lower-risk GBM, said method comprising or consisting of determining a risk score R for a GBM patient, R being defined as follows: R=Σᵢ aᵢ fᵢ wherein i runs from 1 to 4; a₁ is between 0.30 and 0.40, and is preferably 0.33; a₂ is between −0.35 and −0.25, and is preferably −0.28; a₃ is between 0.45 and 0.55, and is preferably 0.51; and a₄ is between −1.00 and −0.90, and is preferably −0.97; and f₁ to f₄ are the expression levels of miRNAs hsa-miR-615-5p, hsa-miR-125a-5p, hsa-let-7a-5p and hsa-let-7b-5p; wherein an average risk score Rₐᵥ is defined as the average of R in GBM; wherein a value of (R−Rₐᵥ) which is greater than zero is indicative of higher-risk GBM, and a value of (R−Rₐᵥ) which is smaller than zero is indicative of lower-risk GBM; and wherein diagnostic methods practised on the human or animal body as well as methods for treatment of the human or animal body by surgery are excluded.

The risk score R is a weighted sum of the expression levels f₁ to f₄ of the four miRNAs according to the invention. While this is not a requirement, it is preferred to scale the expression data prior to calculating the risk score. The scaling step renders the expression levels determined by different methods of measurement comparable. A standard scaling procedure was used which moves the mean value distribution towards 0 by subtracting a so-called Windsorized mean (i.e. mean of all values, but the most extreme values, preferably the 30% lowest and 30% highest values, are excluded) and scaling the bandwidth of the value distribution by division with the standard deviation.

Any of the art-established methods for determining expression levels may be used. These include, but are not confined to microarray-based assays, quantification using RNA-sequencing, and qRT-PCR.

These and other methods are capable of generating (semi-)quantitative expression values of the miRNAs and standard non-noncoding RNAs.

Expression levels are preferably determined from tissue samples. Said tissue samples may be fresh, fresh-frozen or formalin-fixed paraffin-embedded (FFPE) tissue sections.

Alternatively, it is also envisaged to use of body fluids, especially blood, serum or plasma.

For the average of R, the same considerations apply as for the above mentioned reference state and the average expression levels.

Preferred intervals for a₁ are between 0.30 and 0.35 and between 0.32 and 0.34. Preferred intervals for a₂ are between −0.30 and −0.25 and between −0.29 and −0.27. Preferred intervals for a₃ are between 0.50 and 0.55 and between 0.50 and 0.52. Preferred intervals for a₄ are between −1.0 and −0.95 and between −0.98 and −0.96.

Related to the third aspect, the present invention also provides a method of diagnosing higher-risk GBM or lower-risk GBM, said method comprising or consisting of determining a risk score R for a GBM patient, R being defined as follows: R=Σᵢ aᵢ fᵢ, wherein i runs from 1 to 4; a₁ is between 0.30 and 0.40, and is preferably 0.33; a₂ is between −0.35 and −0.25, and is preferably −0.28; a₃ is between 0.45 and 0.55, and is preferably 0.51; and a₄ is between −1.00 and −0.90, and is preferably −0.97; and f₁ to f₄ are the expression levels of miRNAs hsa-miR-615-5p, hsa-miR-125a-5p, hsa-let-7a-5p and hsa-let-7b-5p; wherein an average risk score Rₐᵥ is defined as the average of R in GBM; wherein a value of (R−Rₐᵥ) which is greater than zero is indicative of higher-risk GBM, and a value of (R−Rₐᵥ) which is smaller than zero is indicative of lower-risk GBM.

In a preferred embodiment of the methods according to the second and third aspect of the invention, said method comprises subjecting a sample taken from GBM patient to an analytical method, said analytical method preferably employing the means as defined in accordance with the first aspect.

Preferred analytical methods are the methods for determining expression levels as disclosed herein.

In a preferred embodiment of the methods according to the second and third aspect of the invention, said GBM patient is isocitrate dehydrogenase 1 (IDH1) mutation negative.

In a low proportion of glioblastoma multiforme (GBM) patients (about 5%) there is a mutation of the isocitrate dehydrogenase 1 (IDH1) gene; see Reuss et al. (Acta Neuropathol (2015); 129:133-146). A typical mutation in the human IDH1 protein is R132H. Generally speaking, a IDH1 mutation is typical feature of lower grade (less than WHO grade IV) gliomas. As regards glioblastomas (grade IV) with an IDH1 mutation, it is generally considered the tumors were initially of lower grade and have acquired the mutation in the course of tumorigenesis. Typically, GBM with an IDH1 mutation exhibits better prognosis. On the other hand, GBM with no mutation in the IDH1 gene generally has bad prognosis. As a consequence, there is a particular interest in analyzing, diagnosing and stratifying those GBM patients which do not have a mutation in the IDH1 gene.

In a fourth aspect, the present invention provides a kit as defined in accordance with the first aspect for use in a method of stratifying GBM or a method of diagnosing higher-risk GBM or lower-risk GBM, said method preferably being the method of the second or third aspect, respectively.

Related thereto, the present invention provides in a fifth aspect a use of the kit as defined in the first aspect for stratifying GBM or for diagnosing higher-risk GBM or lower-risk GBM, wherein diagnostic methods practised on the human or animal body as well as methods for treatment of the human or animal body by surgery are excluded, and wherein preferably said diagnosing is effected by the method of the second or third aspect, respectively.

In preferred embodiments of the second, third, fourth and fifth aspect, said higher-risk GBM is not responsive to postoperative radiotherapy.

As noted above, higher-risk GBM, i.e. those forms of GBM which are characterized by bad prognosis, include or consist of forms of GBM which are not responsive to treatment, especially not to post-operative radiotherapy.

Higher-risk vs. lower-risk is also distinguishable in terms of hazard ratio. Hazard ratio is the instantaneous risk of dying relative to a baseline hazard. For the higher-risk group a hazard ratio of 3.79 was found in the discovery cohort, and 2.11 in the validation cohort, respectively. Here the baseline corresponds to the lower-risk group.

In preferred embodiments of the kit according to the first aspect, the kit for use according to the fourth aspect or the use according to the fifth aspect, said kit and/or said means, respectively, comprise(s) or consist(s) of a carrier.

The carrier may be solid carrier or a gel. The carrier may be such that it has a planar surface or it may take the shape of beads. Carriers may be used for the purpose of implementing the kit as mini-array. As will be apparent further below, preferred implementations of the above disclosed means include probes and primers. Means and methods for attaching such means to the surface of a carrier are known in the art.

In further preferred embodiments of the kit, of the kit for use or the use in accordance with the invention, said kit further comprises a manual describing the method of the second or third aspect, respectively.

In a further preferred embodiment of the kit, the kit for use or the use of the kit, said means comprise or consist of probes and/or primers which are specific for the respective miRNA, wherein optionally said probes and/or primers are immobilized on the carrier as defined above.

Preferred lengths of said primers and probes, respectively, are between 15 and 50, preferably between 18 and 30 nucleotides.

Preferred specific forward primers are given in Table 1 below:

TABLE 1
Primers for detecting or quantifying miRNAs in
accordance with the invention.

| | SEQ | |
|---|---|---|
| | ID | |
| miRNA | NO: | sequence |
| hsa-miR-615-5p | 1 | 5′GGGGGUCCCCGGUGCUCGGAUC |
| hsa-miR-125a-5p | 2 | 5′UCCCUGAGACCCUUUAACCUGUGA |
| hsa-let-7a-5p | 3 | 5′UGAGGUAGUAGGUUGUAUAGUU |
| hsa-let-7b-5p | 4 | 5′UGAGGUAGUAGGUUGUGUGGUU |

In a further preferred embodiment of the kit for use or the use of the kit, said stratifying and said diagnosing is to be effected for GBM patients which are IDH1 mutation negative.

In further preferred embodiments of the method in accordance with the second and the third aspect, said determining comprises the use of probes and/or primers which are specific for the respective miRNA, said use preferably being for determining the expression level of the respective miRNAs.

If not expressly indicated otherwise, preferred embodiments of one aspect of the invention apply mutatis mutandis also to other aspects of the invention, i.e. they define also preferred embodiments of said other aspects of the invention.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show:

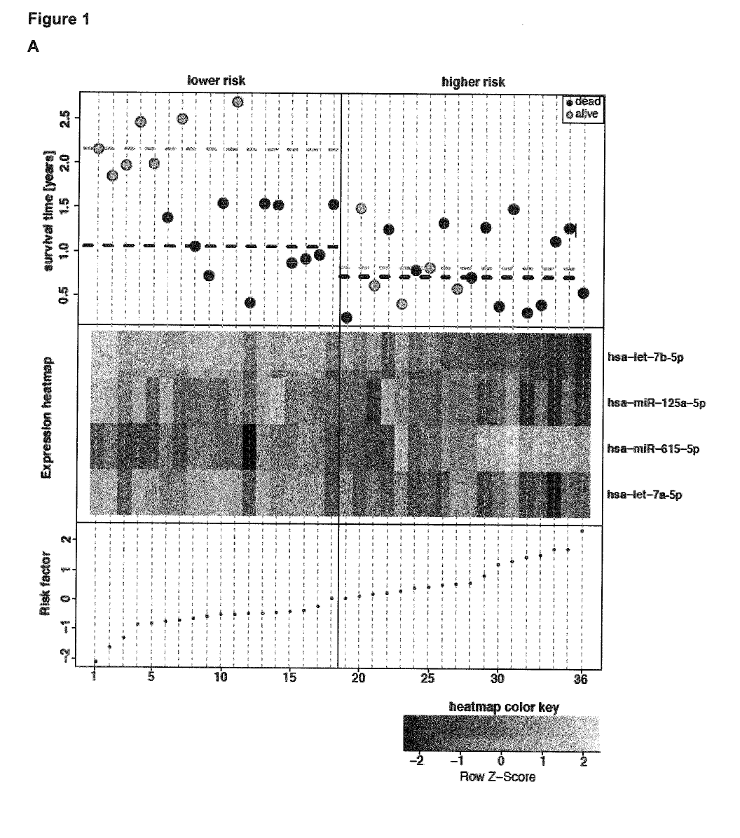

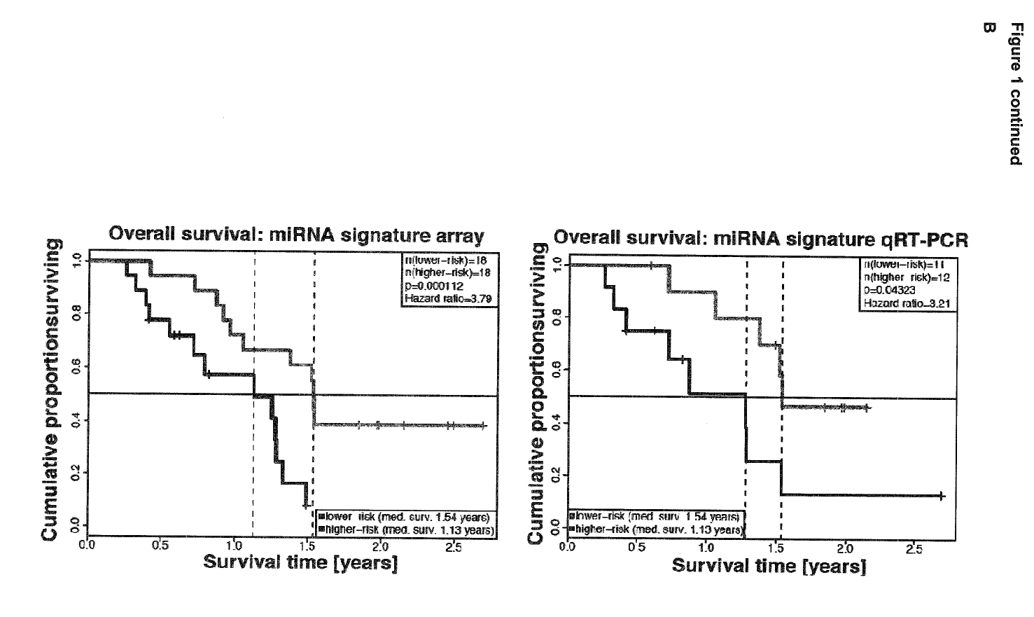

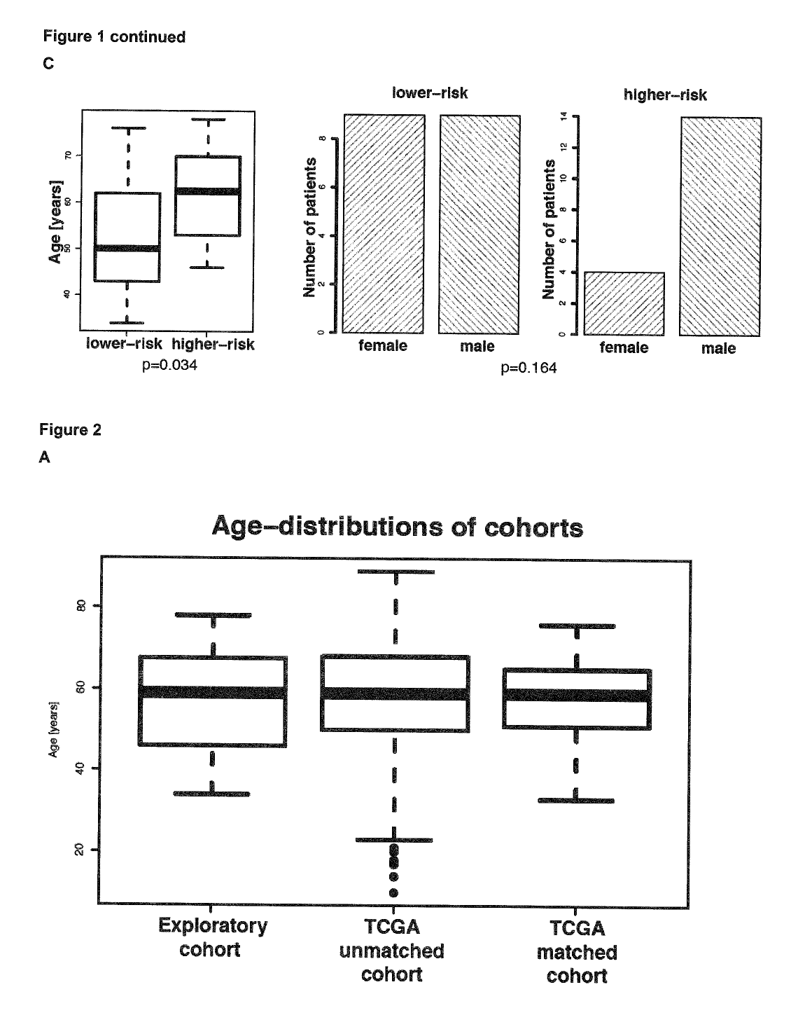

FIG. 1. Extraction of a 4-miRNA signature as independent predictive marker for the overall survival of GBM patients in the exploratory cohort.

(A) Overall survival (top panel), hierarchical cluster heat map of miRNA array expression levels (middle panel), and risk factors calculated on the basis of miRNA expression values and Coxph coefficients (bottom panel) for all patients. The median risk factor value was used to classify higher-risk and lower-risk patients.

(B) Kaplan-Meier overall survival analyses of higher-risk and lower-risk GBM patients. Higher-risk and lower-risk patients were stratified based on the risk factors calculated from the Coxph coefficients and the miRNA expression levels as measured in the microarray (left panel, 35 patients) or by qRT-PCR analyses (right panel, 19 patients). Hazard ratios and p-values were calculated by log-rank test. (C) Distribution of age (left panel) and sex (middle and right panels) in higher-risk and lower-risk GBM patients. Statistical comparison was performed by Student's t-test and Fisher's exact, respectively.

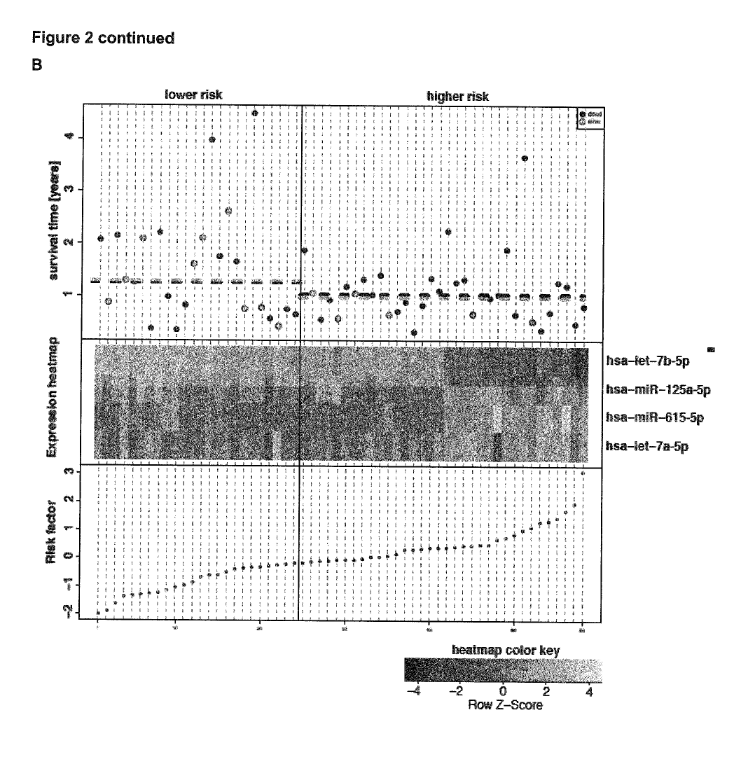

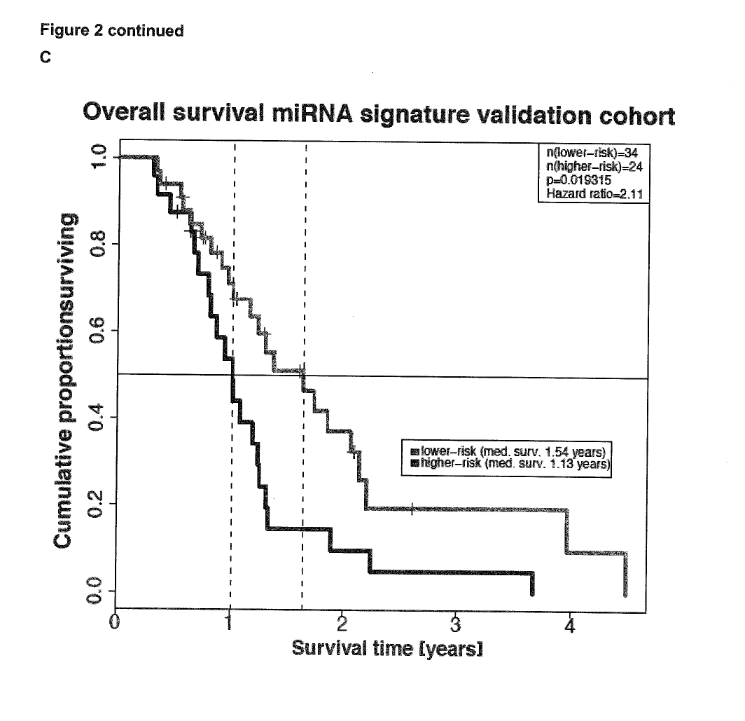

FIG. 2. Evaluation of the prognostic value of the extracted 4-miRNA signature in an age- and sex-matched subgroup of the TCGA GBM dataset.

(A) Age distribution in the exploratory cohort and the TCGA GBM cohort before and after age matching. (B) Overall survival (top panel), hierarchical cluster heat map of miRNA expression levels (middle panel), and risk factors for patients of the age- and sex-matched TOGA GBM cohort. The median risk factor value was used to classify higher-risk and lower-risk patients. (C) Kaplan-Meier overall survival analyses of higher-risk and lower-risk patients of the age- and sex-matched TOGA GBM cohort. Classification of higher-risk and lower-risk patients was performed on the basis of the risk factors calculated from the Coxph coefficients (Tab. 1) and the miRNA expression levels. Hazard ratios and p-values were calculated by log-rank test. (D) Distribution of age (left panel) and sex (middle and right panel) in higher-risk and lower-risk patients of the age- and sex-matched TOGA GBM cohort. Student's t-test and Fisher's exact test were employed for statistical comparisons.

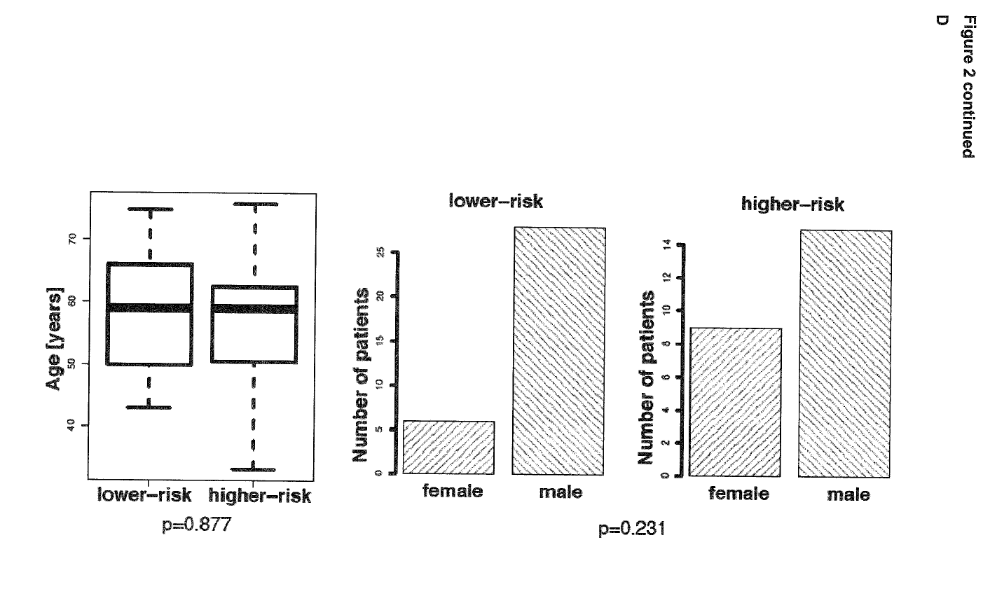

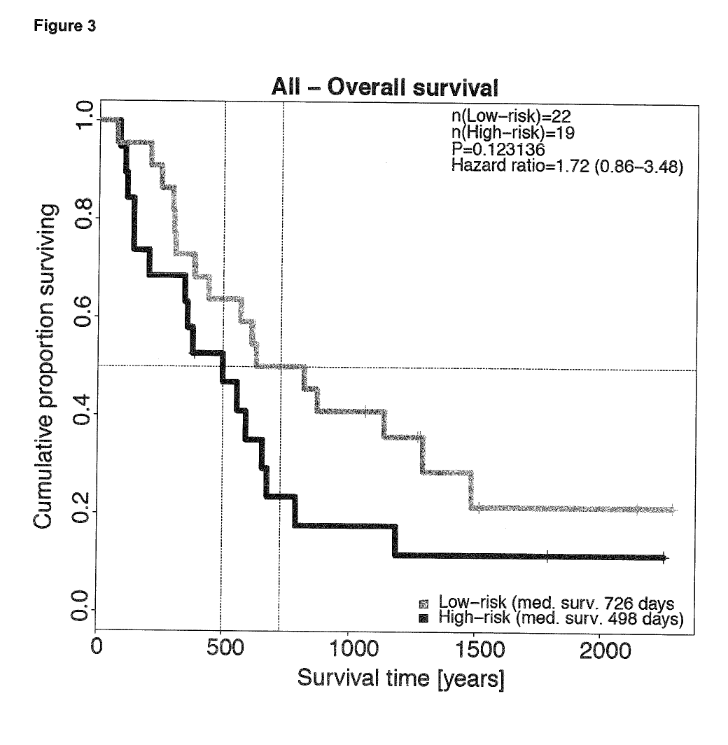

FIG. 3. Kaplan-Meier plot of high-risk (red) and low-risk (blue) patients defined using the 4-miRNA signature of the invention.

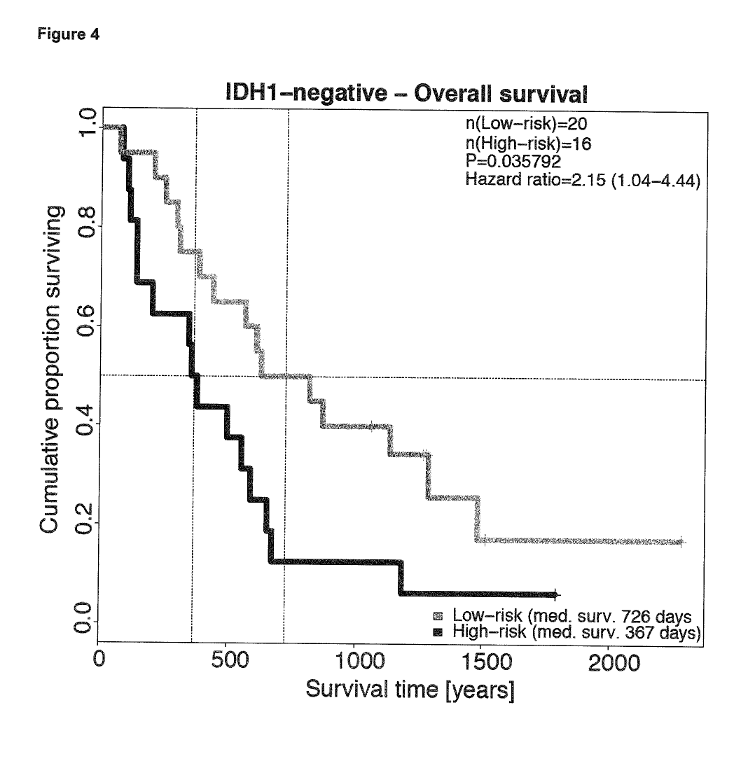

FIG. 4. Kaplan-Meier plot of high-risk (red) and low-risk (blue) patients of the IDH1 mutation negative subgroup defined using the 4-miRNA signature of the invention.

The examples illustrate the invention

### EXAMPLE 1

Low Complexity miRNA Signature and Evaluation of its Prognostic Significance for Overall Survival

A signature that consisted of the four miRNAs hsa-let-7a-5p, hsa-let-7b-5p, hsa-miR-125a-5p and hsamiR-615-5p which was statistically significantly associated with overall survival (p-value=0.0048), was found. The median risk score calculated from the expression levels of the signature miRNAs and the corresponding coxph coefficients (Table 2) separated the patients into a high- and a lower-risk group.

TABLE 2
Cox-proportional hazard coefficients used in risk score calculation
Table 2

| miRNA | coefficient |
|---|---|
| hsa-let-7b-5p | −0.9669152 |
| hsa-miR-125a-5p | −0.2821517 |
| hsa-miR-615-5p | 0.3254795 |
| hsa-let-7a-5p | 0.5059587 |

Cox regression analyses of the high- and the lower-risk groups revealed a 5.11 fold increased risk of death (95% CI: 2.03-12.85) for the higher-risk group compared to the lower-risk group (p-value=0.000112). The median survival time was 13.5 months for patients of the higher-risk group and 18.4 months for patients of the lower-risk group, respectively. These results were visualized by Kaplan-Meier overall survival curves (FIG. 1B). Univariate testing of the individual miRNAs within the signature revealed p-values in the range between 0.0015 and 0.016, indicating that each single miRNA was able to statistically significantly predict overall survival. FIG. 1A summarizes the survival data of the patients in relation to the calculated risk scores and expression levels.

### EXAMPLE 2

Independent in Silico Validation of the Detected miRNA Signature

For the purpose of independent validation the miRNA signature was tested in an age-matched miRNA data subset of an independent GBM cohort downloaded from the TCGA database (Cancer Genome Atlas Research Network. Comprehensive genomic characterization defines human glioblastoma genes and core pathways Nature. 2008; 455:1061-8). The high- and the lower-risk groups were defined by using the median risk score of the discovery set to dichotomize the patients of the validation set. The resulting coxph model revealed a hazard ratio of 2.11 (95% CI 1.13-3.91) and a p-value of 0.019 (FIG. 2D). FIG. 2B summarizes the survival data of the patients of the validation cohort in relation to calculated risk scores and expression levels.

### EXAMPLE 3

Technical Validation of Signature by qRT-PCR

In order to technically validate the 4-miRNA signature and to support potential applicability in clinical routine diagnostics, we measured the expression of the four miRNAs in a subset of samples (n=23), for which residual material was available by qRT-PCR. Analogous cox proportional hazard analysis with the qRT-PCR data confirmed the results obtained with the miRNA array data. Patients of the higher-risk group revealed significantly impaired overall survival (p-value=0.043) and a hazard-ratio of 3.21 (95% CI 1.02-10.16) as compared to patients of the lower-risk group. (FIG. 1B).

### EXAMPLE 4

### Validation in Independent Cohort

### Material and Methods

### Patients Cohort

A retrospective cohort of 41 patients who were treated according the Stupp et al. (Lancet Oncol (2009); 10:459-466) standard-of-care protocol was recruited from the LMU Neuropathology and Radiation Oncology Departments' registries. 17% of patients were younger and 83% were older than 50 years with a median age of 64.5 years. 46% of patients were male and 54% were female. Median follow-up time was 1.61 years (95% CI: 1.05-2.38). 5 out of the 41 patients were insocitrate dehydrogenase 1 (IDH1) mutation positive. With regard to MGMT promoter methylation status 10 patients were MGMT-promoter methylation negative and 31 were positive.

### RNA Isolation

From the formalin-fixed paraffin-embedded tissue blocks that were prepared from resected tumours 5 micron HE reference slides were used to identify areas of tumour tissue with >80% tumour cell content that were used for macrodissection of five serial 10 micron sections. The macrodissected tissue was subjected to RNA isolation after deparaffinisation as described in Niyazi et al. (Oncotarget (2016); 7:45764-45775).

### Quantitative Real-Time PCR

Total RNA containing the small noncoding RNA fraction was subjected to SYBR green chemistry based qRT-PCR as described in Niyazi et al. (2016). The measured median CT values of the signature miRNAs were transformed to delta Cts by subtracting CTs of the reference small non-coding RNA SNORD68 per sample. The inverted values were used as expression values and the resulting expression matrix was scaled per miRNA.

### Calculation of Risk Scores

Using the cox-proportional hazard coefficients from the initial signature model as published in the Niyazi et al. study risk scores were calculated for each patient. The patients were assigned to the high- and low-risk group using the threshold published in the Niyazi et al. (2016) study followed by Kaplan-Meier analysis for the calculation of the log-rank score statistics and hazard-ratio.

### Results

In the complete group of patients (n=41) a trend of high-risk patients with worse survival compared to low-risk patients was obvious (FIG. 3). When excluding patients bearing an IDH1 mutation the difference in overall survival between high-risk and low-risk patients is statistically significant (p-value: 0.036, hazard-ratio: 2.15 (95%-CI: 1.05-4.44), FIG. 4). Further, univariate and multivariate analysis showed that the signature risk score in the investigated cohort is independent of the risk factors age, sex, MGMT methylation.

### Discussion

In the IDH1 mutation negative subgroup of patients the difference in overall survival between high-risk and low-risk patients was statistically significant. This is an important finding since IDH1 mutation positive GBM patients (secondary GBMs) are known to have a much better prognosis compared with IDH1 mutation negative ones (de novo GBMs). Thus, from a clinical perspective, IDH1 mutation negative GBM patients build the clinical relevant group for a personalised risk-stratified treatment. Moreover, from a pathological point of view IDH1 mutation positive GBM are considered being secondary GBMs emerging from lower-grade gliomas (Reuss (2015)). Therefore, IDH1 mutation positive tumours comprise a different etiology of gliomas which is also reflected in the prognosis of affected patients (Cai et al., Oncoscience (2016); 3:258-265).

We were able to validate the 4-miRNA signature of the invention in the IDH1 mutation negative subgroup of a second independent cohort of patients. This finding substantiates the prognostic value of the signature and provides further evidence for its validity.

1. A kit
(a) comprising or
(b) consisting of
means for detecting and/or quantifying one, two, three or all four of the following miRNAs: hsa-miR-615-5p; hsa-miR-125a-5p; hsa-let-7a-5p; and hsa-let-7b-5p.

2. The kit of claim 1(a), wherein said kit comprises no means for detecting and/or quantifying miRNAs or RNAs other than one, two, three or all four of hsa-miR-615-5p; hsa-miR-125a-5p; hsa-let-7a-5p; and hsa-let-7b-5p, wherein said kit optionally comprises means for quantifying one or more standard RNAs.

3. A method of stratifying glioblastoma multiforme (GBM),
said method comprising or consisting of determining for a GBM patient one, two, three or all four of the expression levels of miRNAs hsa-miR-615-5p, hsa-miR-125a-5p, hsa-let-7a-5p and hsa-let-7b-5p,
wherein
(a) the more the expression level of hsa-miR-615-5p is increased and/or the more the expression levels of hsa-miR-125a-5p, hsa-let-7a-5p and hsa-let-7b-5p are decreased, the worse is the prognosis of said patient;
(b) determining whether an expression level is increased or decreased is by comparison with a reference state, said reference state being the average expression levels in GBM of hsa-miR-615-5p, hsa-miR-125a-5p, hsa-let-7a-5p and hsa-let-7b-5p, respectively; and
(c) diagnostic methods practised on the human or animal body as well as methods for treatment of the human or animal body by surgery are excluded.

4. The method of claim 3, wherein
(i) an expression level of hsa-miR-615-5p which is increased and/or expression levels of hsa-miR-125a-5p, hsa-let-7a-5p and hsa-let-7b-5p which are decreased is/are indicative of bad prognosis; and
(ii) an expression level of hsa-miR-615-5p which is decreased and/or expression levels of hsa-miR-125a-5p, hsa-let-7a-5p and hsa-let-7b-5p which are increased is/are indicative of good prognosis.

5. The method of claim 3 or 4, wherein said method does not comprise assessing expression levels of miRNAs or RNAs other than one, two, three or all four of hsa-miR-615-5p; hsa-miR-125a-5p; hsa-let-7a-5p; and hsa-let-7b-5p, wherein said method optionally comprises quantifying the expression level(s) of one or more standard RNAs.

6. A method of diagnosing higher-risk GBM or lower-risk GBM, said method comprising or consisting of determining a risk score R for a GBM patient, R being defined as follows: R=Σᵢ aᵢ fᵢ
wherein
i runs from 1 to 4;
a₁ is between 0.30 and 0.40, and is preferably 0.33; a₂ is between −0.35 and −0.25, and is preferably −0.28; a₃ is between 0.45 and 0.55, and is preferably 0.51; and a₄ is between −1.00 and −0.90, and is preferably −0.97; and
f₁ to f₄ are the expression levels of miRNAs hsa-miR-615-5p, hsa-miR-125a-5p, hsa-let-7a-5p and hsa-let-7b-5p;
wherein an average risk score Rₐᵥ is defined as the average of R in GBM;
wherein a value of (R−Rₐᵥ) which is greater than zero is indicative of higher-risk GBM, and a value of (R−Rₐᵥ) which is smaller than zero is indicative of lower-risk GBM; and
wherein diagnostic methods practised on the human or animal body as well as methods for treatment of the human or animal body by surgery are excluded.

7. The method of any one of claims 3 to 6, wherein said method comprises subjecting a sample taken from GBM patient to an analytical method, said analytical method preferably employing the means as defined in claims 1 and 2.

8. The method of any one of claims 3 to 7, wherein said GBM patient is isocitrate dehydrogenase 1 (IDH1) mutation negative.

9. A kit of claim 1 or 2 for use in a method of stratifying GBM or a method of diagnosing higher-risk GBM or lower-risk GBM, said method preferably being the method of any one of claims 3 to 7.

10. Use of the kit of claim 1 or 2 for stratifying GBM or for diagnosing higher-risk GBM or lower-risk GBM, wherein diagnostic methods practised on the human or animal body as well as methods for treatment of the human or animal body by surgery are excluded, and wherein preferably said diagnosing is effected by the method of any one of claims 3 to 7.

11. The method of any one of claims 3 to 8, the kit for use of claim 9, or the use of claim 9, wherein said higher-risk GBM is not responsive to postoperative radiotherapy.

12. The kit of claim 1 or 2, the kit for use of claim 9 or 11, or the use of claim 10 or 11, wherein said kit and/or said means, respectively, comprise(s) or consist(s) of a carrier.

13. The kit of any one of claim 1, 2 or 12, or the kit for use of any one of claim 9, 11 or 12, or the use of any one of claims 10 to 12, said kit further comprising a manual describing the method of any one of claim 3 to 8 or 11.

14. The kit, kit for use or use of any of the preceding claims, wherein said means comprise or consist of probes and/or primers which are specific for the respective miRNA, wherein optionally said probes and/or primers are immobilized on the carrier as defined in claim 12.

15. The kit for use or use of any of the preceding claims, wherein said stratifying and said diagnosing is to be effected for GBM patients which are IDH1 mutation negative.

16. The method of any of claim 3 to 8 or 11, wherein said determining comprises the use of probes and/or primers which are specific for the respective miRNA, said use preferably being for determining the expression level of the respective miRNAs.
